(19) United States
(12) Patent Application Publication
(10) Pub. No.: US 2021/0215701 A1
(43) Pub. Date: Jul. 15, 2021

(54) COMPOSITIONS FOR OVARIAN CANCER ASSESSMENT

(71) Applicant: Aspira Women's Health Inc., Austin, TX (US)

(72) Inventors: Charles Dunton, Austin, TX (US); Rowan Bullock, Austin, TX (US); Herbert Fritsche, Austin, TX (US)

(73) Assignee: Aspira Women's Health Inc., Austin, TX (US)

(21) Appl. No.: 17/267,947

(22) PCT Filed: Aug. 13, 2019
(86) PCT No.: PCT/US19/46317

§ 371 Date: Feb. 11, 2021

Related U.S. Application Data
(60) Provisional application No. 62/718,198, filed on Aug. 13, 2018.

(51) Int. Cl. G01N 33/574; C12Q 1/6804

(52) U.S. Cl. CPC G01N 33/57449; C12Q 1/6804; C12Q 2600/156; C12Q 2600/118; C12Q 2600/158; G01N 2800/50

(57) ABSTRACT

The present invention provides compositions and methods that provide a high degree of sensitivity and a high degree of specificity for the preoperative assessment of ovarian tumors in an African American subject (e.g., pre- and post-menopausal women) having a variety of ovarian cancer types (e.g., clear cell/mucinous, low malignant potential, high malignant potential) and at a variety of disease states (e.g., early and late stage).

### CROSS-REFERENCE TO RELATED APPLICATION

This application claims the benefit of and priority to U.S. Provisional patent Application Ser. No. 62/718,198 filed Aug. 13, 2018, the entire contents of which are hereby incorporated by reference herein.

### BACKGROUND OF THE INVENTION

Ovarian cancer is among the most lethal gynecologic malignancies in developed countries. Annually in the United States alone, approximately 23,000 women are diagnosed with the disease and more than 15,000 women die from ovarian cancer annually. Despite progress in cancer therapy, ovarian cancer mortality has remained virtually unchanged over the past two decades. Although African American women are diagnosed less frequently than their Caucasian counterparts, 5-year survival rates are much lower. Given the steep survival gradient relative to the stage at which the disease is diagnosed, early detection remains the most important factor in improving long-term survival of ovarian cancer patients. A second important factor is whether or not women with ovarian cancer are treated by a surgeon that specializes in gynecological oncology. Improved methods for detecting ovarian cancer and referring them to a surgeon for treatment are urgently required, particularly for African American women.

### SUMMARY OF THE INVENTION

The present invention provides compositions and methods that provide a high degree of sensitivity and a high degree of specificity for the preoperative assessment of ovarian tumors in a variety of African American subject's (e.g., pre- and post-menopausal women) having a variety of ovarian cancer types (e.g., clear cell/mucinous, low malignant potential, high malignant potential) and at a variety of disease states (e.g., early and late stage). In particular embodiments, compositions and methods of the invention are surprisingly effective in reducing the rate of false positive diagnoses of ovarian cancer by 25%, 50%, 66%, 75% or more.

In one aspect, the invention provides a panel for pre-operatively assessing an African American subject's risk of having ovarian cancer, the panel comprising markers:

(a) Transthyretin (TT or prealbumin), Apolipoprotein A-1 (Apo A-1), beta 2-Microglobulin (beta 2M), Transferrin (Tfr) and Cancer Antigen 125 (CA 125); and a marker of ethnicity;

(b) apolipoprotein A-1, CA 125, follicle stimulating hormone, human epididymis protein 4, and transferrin and a marker of ethnicity; or

(c) CA 125 and a marker of ethnicity; or a set of capture reagent each of which specifically binds a marker. In one embodiment, the capture reagent is an antibody that selectively binds a marker.

In another aspect, the invention provides a method for pre-operatively assessing an African American subject's risk of having ovarian cancer, the method involves detecting binding of a capture reagent to a marker selected from:

(a) Transthyretin (TT or prealbumin), Apolipoprotein A-1 (Apo A-1), beta 2-Microglobulin (beta 2M), Transferrin (Tfr) and Cancer Antigen 125 (CA 125); and a marker of ethnicity;

(b) apolipoprotein A-1, CA 125, follicle stimulating hormone, human epididymis protein 4, and transferrin and a marker of ethnicity; or

(c) CA 125 and a marker of ethnicity. In one embodiment, the detecting is by immunoassay or affinity capture. In another embodiment, the immunoassay involves affinity capture assay, immunometric assay, heterogeneous chemiluminscence immunometric assay, homogeneous chemiluminscence immunometric assay, ELISA, western blotting, radioimmunoassay, magnetic immunoassay, real-time immunoquantitative PCR (iqPCR) and SERS label free assay. In another embodiment, the method is carried out in a plate, chip, beads, microfluidic platform, membrane, planar microarray, or suspension array.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

A “biomarker” or “marker” as used herein generally refers to a protein, nucleic acid molecule, clinical indicator, or other analyte or condition that is associated with a disease or the propensity to develop a disease (e.g., ovarian cancer). In one embodiment, the marker is an indicator of ethnicity or genetic ancestry. In one embodiment, a marker of ovarian cancer is differentially present in a biological sample obtained from a subject having or at risk of developing ovarian cancer relative to a reference. A marker is differentially present if the mean or median level of the biomarker present in the sample is statistically different from the level present in a reference. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing ovarian cancer, for determining the prognosis of the subject, for evaluating therapeutic efficacy, or for selecting a treatment regimen (e.g., selecting that the subject be evaluated and/or treated by a surgeon that specializes in gynecologic oncology).

Markers useful in the panels of the invention include, for example, FSH, HE4, CA125, transthyretin, transferrin, ApoA1, β2 microglobulin and CA72-4 proteins, as well as the nucleic acid molecules encoding such proteins, and other markers of ethnicity. Fragments useful in the methods of the invention are sufficient to bind an antibody that specifically recognizes the protein from which the fragment is derived. Markers of ethnicity are described, for example, by Huang et al., BMC Genomics 16: 1093, 2015; by Sampson et al., Ann Hum Genet. 2011 July; 75(4): 539-553; Kidd et al., Forensic Science International. 164 (1): 20-32, 2006. The invention includes markers that are substantially identical to the following sequences. Preferably, such a sequence is at least 85%, 90%, 95% or even 99% identical at the amino acid level or nucleic acid to the sequence used for comparison.

By “Follicle-stimulating hormone (FSH) polypeptide” is meant a polypeptide or fragment thereof having at least about 85% amino acid identity to NCBI Accession No. NP_000501.

By “Human Epididymis Protein 4 (HE4) polypeptide” is meant a polypeptide or fragment thereof having at least about 85% amino acid identity to NCBI Accession No. NP_006094.

By “Cancer Antigen 125 (CA 125) polypeptide” is meant a polypeptide or fragment thereof having at least about 85% amino acid identity to Swiss-Prot Accession number Q8WXI7.

By “Transthyretin (Prealbumin) polypeptide” is meant a polypeptide or fragment thereof having at least about 85% amino acid identity to Swiss Prot Accession number P02766.

By “Transferrin polypeptide” is meant a polypeptide or fragment thereof having at least about 85% amino acid identity to UniProtKB/TrEMBL Accession number Q06AH7.

By “Apolipoprotein A1 (ApoA1) polypeptide” is meant a polypeptide or fragment thereof having at least about 85% amino acid identity to Swiss Prot Accession number P02647.

By “β-2 microglobulin polypeptide” is meant a polypeptide or fragment thereof having at least about 85% amino acid identity to SwissProt Accession No. P61769.

By “agent” is meant any small molecule chemical compound, antibody, nucleic acid molecule, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

By “capture reagent” is meant a reagent that specifically binds a nucleic acid molecule or polypeptide to select or isolate the nucleic acid molecule or polypeptide. In one embodiment, the capture reagent is an antibody or a nucleic acid molecule that hybridizes to a marker polynucleotide.

By “clinical aggressiveness” is meant the severity of the neoplasia. Aggressive neoplasias are more likely to metastasize than less aggressive neoplasias. While conservative methods of treatment are appropriate for less aggressive neoplasias, more aggressive neoplasias require more aggressive therapeutic regimens.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

By “Marker profile” is meant a characterization of the expression or expression level of two or more polypeptides or polynucleotides.

The term “ovarian cancer” refers to both primary ovarian tumors as well as metastases of the primary ovarian tumors that may have settled anywhere in the body.

The term “ovarian cancer status” refers to the status of the disease in the patient. Examples of types of ovarian cancer statuses include, but are not limited to, the subject's risk of cancer, the presence or absence of disease, the stage of disease in a patient, and the effectiveness of treatment of disease. In embodiments, a subject identified as having a pelvic mass is assessed to identify if their ovarian cancer status is benign or malignant.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “reference” is meant a standard of comparison. For example, the marker level(s) present in a patient sample may be compared to the level of the marker in a corresponding healthy cell or tissue or in a diseased cell or tissue (e.g., a cell or tissue derived from a subject having ovarian cancer). In particular embodiments, the FSH, HE4, CA 125; Transthyretin, Transferrin, polypeptide level present in a patient sample may be compared to the level of said polypeptide present in a corresponding sample obtained at an earlier time point (i.e., prior to treatment), to a healthy cell or tissue or a neoplastic cell or tissue that lacks a propensity to metastasize. As used herein, the term “sample” includes a biologic sample such as any tissue, cell, fluid, or other material derived from an organism.

By “specifically binds” is meant a compound (e.g., antibody) that recognizes and binds a molecule (e.g., polypeptide), but which does not substantially recognize and bind other molecules in a sample, for example, a biological sample.

The accuracy of a diagnostic test can be characterized using any method well known in the art, including, but not limited to, a Receiver Operating Characteristic curve (“ROC curve”). An ROC curve shows the relationship between sensitivity and specificity. Sensitivity is the percentage of true positives that are predicted by a test to be positive, while specificity is the percentage of true negatives that are predicted by a test to be negative. An ROC is a plot of the true positive rate against the false positive rate for the different possible cutpoints of a diagnostic test. Thus, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect test. In embodiments, biomarkers and diagnostic methods of the present invention have an AUC greater than 0.50, greater than 0.60, greater than 0.70, greater than 0.80, or greater than 0.9.

Other useful measures of the utility of a test are positive predictive value (“PPV”) and negative predictive value (“NPV”). PPV is the percentage of actual positives who test as positive. NPV is the percentage of actual negatives that test as negative.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### BRIEF DESCRIPTION OF THE DRAWINGS

FIG. 1 is a table showing cancer subtype as a function of race, and showing the sensitivity of OVA1 for detection of ovarian cancer.

FIG. 2 provides the sequences of the markers referenced in the application.

### DETAILED DESCRIPTION OF THE INVENTION

The invention comprises panels of biomarkers and the use of such panels for characterizing ovarian cancer.

The invention further features the use of such panels for characterizing ovarian cancer (e.g., assessing risk of malignancy, diagnosis, prognosis). In particular, the use of such panels provides methods for pre-surgically characterizing a pelvic mass in a subject and identifying subjects that might benefit from evaluation/treatment by a gynecological oncologist.

### Ovarian Cancer

Ovarian tumors are being detected with increasing frequency in women of all ages, yet there is no standardized or reliable method to determine which are malignant prior to surgery. In 1994, the National Institutes of Health (NIH) released a consensus statement indicating that women with ovarian masses having been identified preoperatively as having a significant risk of ovarian cancer should be given the option of having their surgery performed by a gynecologic oncologist. At present, the National Comprehensive Cancer Network (NCCN), the Society of Gynecologic Oncologists (SGO), SOGC clinical practice guidelines, Standing Subcommittee on Cancer of the Medical Advisory Committee, and several other published statements, all recommend that women with ovarian cancer be under the care of a gynecologic oncologist (GO).

Recent publications on breast, bladder, gastrointestinal, and ovarian cancers have reported improved outcome when cancer management involves a surgical specialist. In addition, a recent meta-analysis of 18 ovarian cancer studies found that the early involvement of a gynecologic oncologist, rather than a general surgeon or general gynecologist, improved patient outcomes. The authors concluded: 1) subjects with early stage disease are more likely to have comprehensive surgical staging, facilitating appropriate adjuvant chemotherapy, 2) subjects with advanced disease are more likely to receive optimal cytoreductive surgery, and 3) subjects with advanced disease have an improved median and overall 5-year survival. Despite the availability of this important information, only a fraction of women with malignant ovarian tumors (an estimated 33%) are referred to a gynecologic oncologist for the primary surgery. Based on reported patterns of care for ovarian cancer management, the majority of women in the United States may not be receiving optimal care for this disease.

The decision for operative removal of an ovarian tumor, and whether a generalist or specialist should perform the surgery, is based on interpretations of physical examination, imaging studies, laboratory tests, and clinical judgment. Pelvic examination alone is inadequate to reliably detect or differentiate ovarian tumors, particularly in early stages when ovarian cancer treatment is most successful. Examination has also been eliminated from the Prostate, Lung, Colorectal and Ovarian cancer screening trial algorithm. Pelvic ultrasound is clinically useful and the least expensive imaging modality, but has limitations in consistently identifying malignant tumors. In general, nearly all unilocular cysts are benign, whereas complex cystic tumors with solid components or internal papillary projections are more likely to be malignant. CA 125 has been used alone or in conjunction with other tests in an effort to establish risk of malignancy. Unfortunately, CA 125 has low sensitivity (50%) in early stage ovarian cancers, and low specificity resultant from numerous false positives in both pre- and postmenopausal women.

The American College of Obstetrics and Gynecology (ACOG) and the SGO have published referral guidelines for patients with a pelvic mass. These guidelines include: patient age, serum CA 125 level, physical examination, imaging results, and family history. This referral strategy has been evaluated both retrospectively and prospectively. In a single institution review, Dearking and colleagues concluded that the guidelines were useful in predicting advanced stage ovarian cancer, but “performed poorly in identifying early-stage disease, especially in premenopausal women, primarily due to lack of early markers and signs of ovarian cancer”.

### Biomarkers

In particular embodiments, a biomarker is an organic biomolecule that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for characterizing a disease.

### Biomarkers for Ovarian Cancer

The invention provides a panel of polypeptide biomarkers that are differentially present in subjects having ovarian cancer, in particular, a benign vs. malignant pelvic mass. The biomarkers of this invention are differentially present depending on ovarian cancer status, including, subjects having ovarian cancer vs. subjects that do not have ovarian cancer.

The biomarker panel of the invention comprises one or more of the biomarkers presented in the following Table 1.

TABLE 1

| | Differential Regulation |
|---|---|
| Biomarker | in ovarian cancer |
| ApoAl | Decreased |
| Beta2 Microglobulin (B2M) | Increased |
| Follicle-stimulating | Increased |
| hormone (FSH) | |
| Human Epididymis | Increased |
| Protein 4 (HE4) | |
| Cancer Antigen 125 (CA 125) | Increased |
| Transthyretin | Decreased |
| (prealbumin) | |
| Transferrin | Decreased |

As would be understood, references herein to a biomarker of Table 1, a panel of biomarkers, or other similar phrase indicates one or more of the biomarkers set forth in Table 1 or otherwise described herein.

A biomarker of the invention may be detected in a biological sample of the subject (e.g., tissue, fluid), including, but not limited to, blood, blood serum, plasma, saliva, urine, ascites, cyst fluid, a homogenized tissue sample (e.g., a tissue sample obtained by biopsy), a cell isolated from a patient sample, and the like.

The invention provides panels comprising isolated biomarkers. The biomarkers can be isolated from biological fluids, such as urine or serum. They can be isolated by any method known in the art. In certain embodiments, this isolation is accomplished using the mass and/or binding characteristics of the markers. For example, a sample comprising the biomolecules can be subject to chromatographic fractionation and subject to further separation by, e.g., acrylamide gel electrophoresis. Knowledge of the identity of the biomarker also allows their isolation by immunoaffinity chromatography. By “isolated biomarker” is meant at least 60%, by weight, free from proteins and naturally-occurring organic molecules with which the marker is naturally associated. Preferably, the preparation is at least 75%, more preferably 80, 85, 90 or 95% pure or at least 99%, by weight, a purified marker.

### Follicle-Stimulating Hormone (FSH)

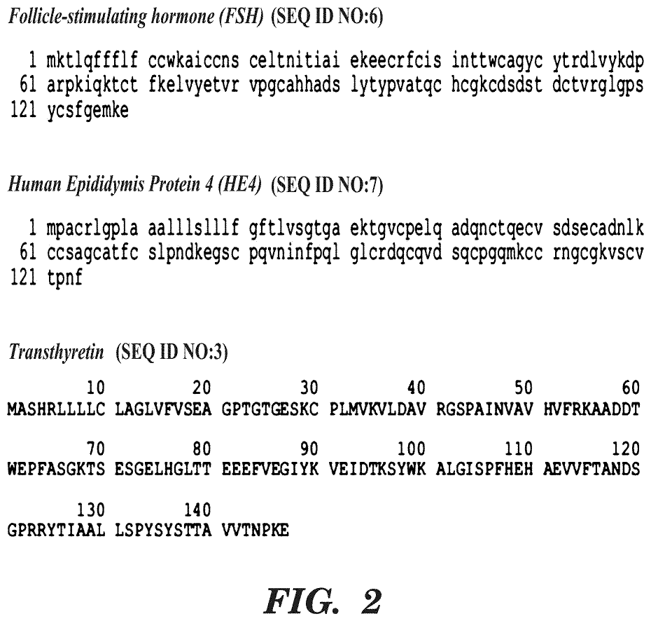

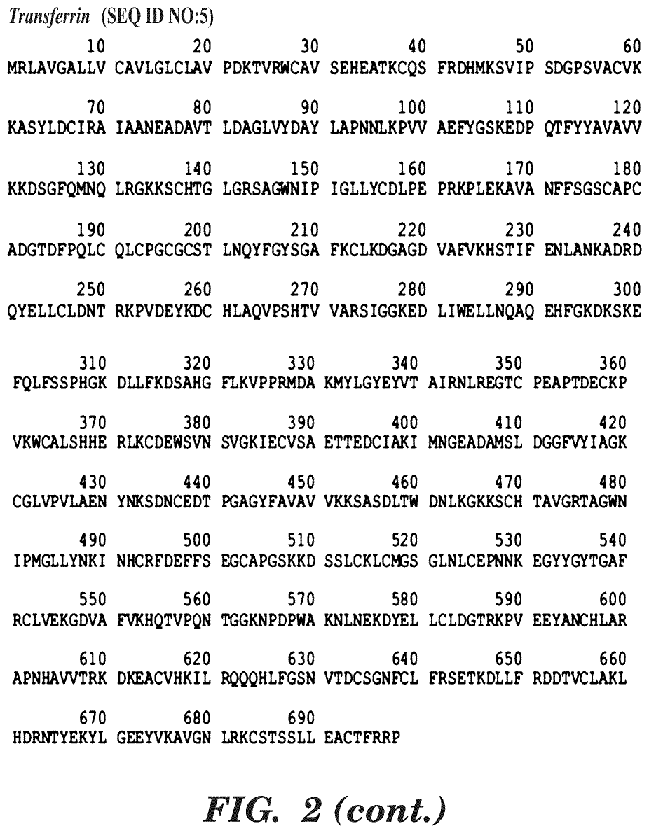

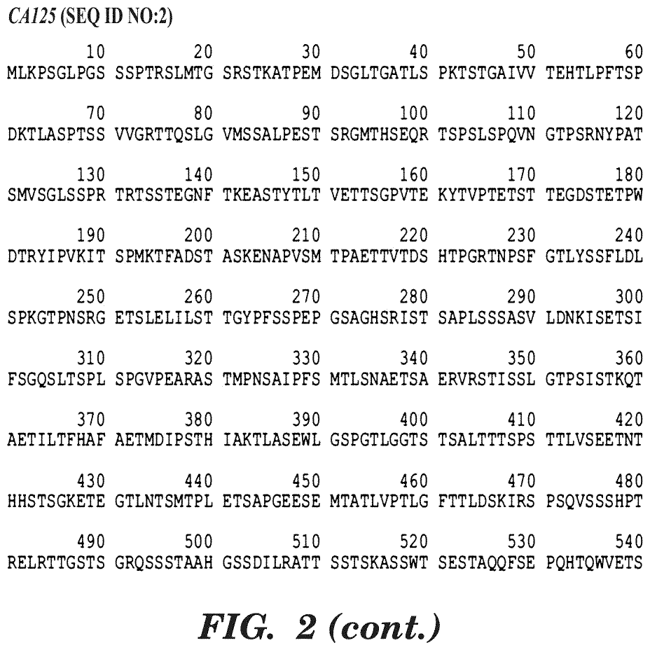

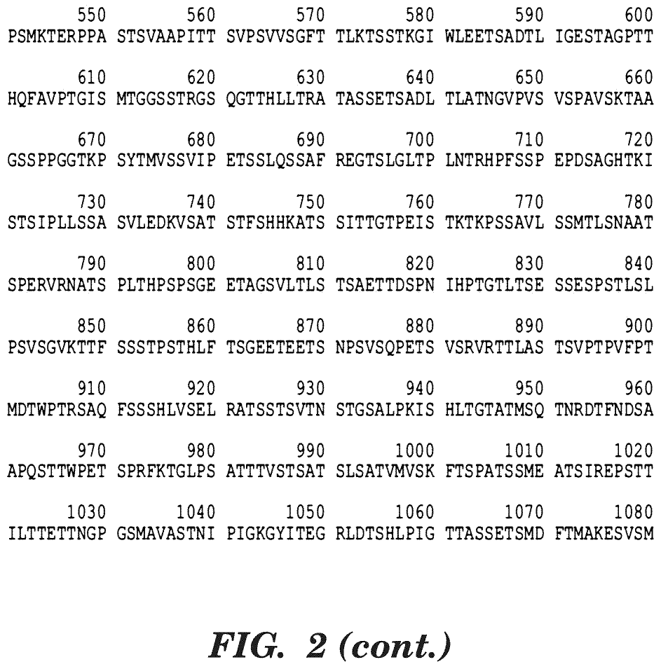

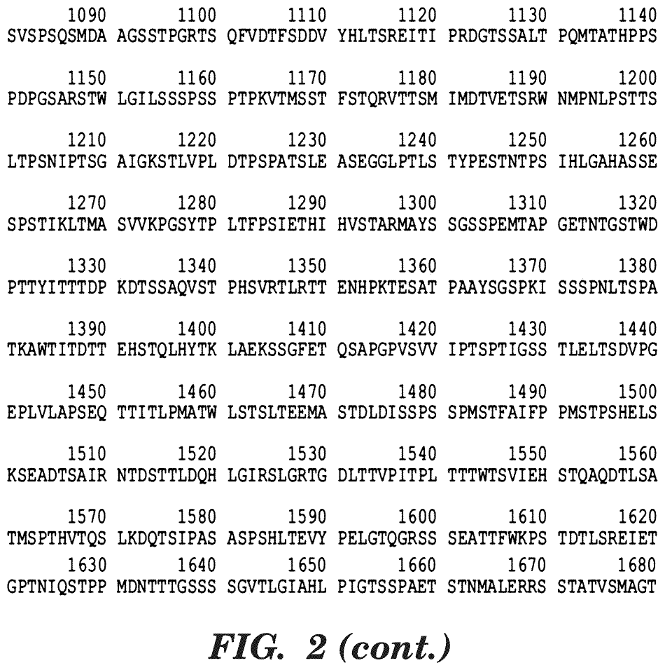

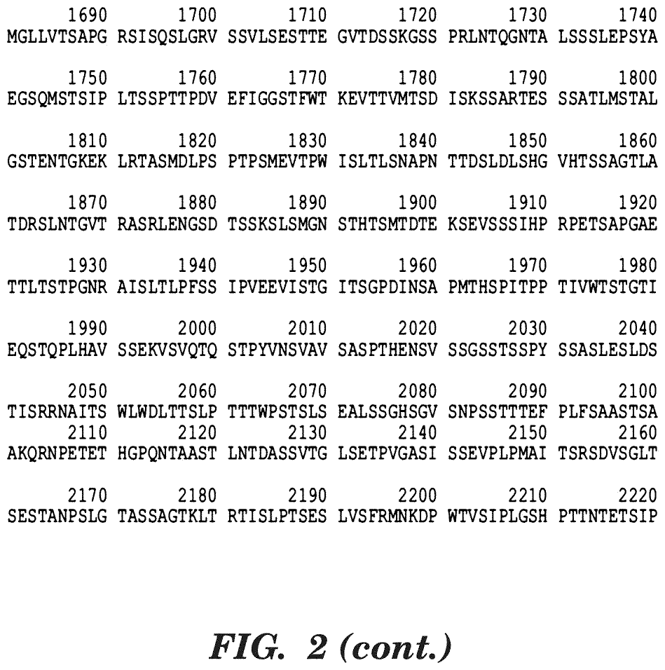

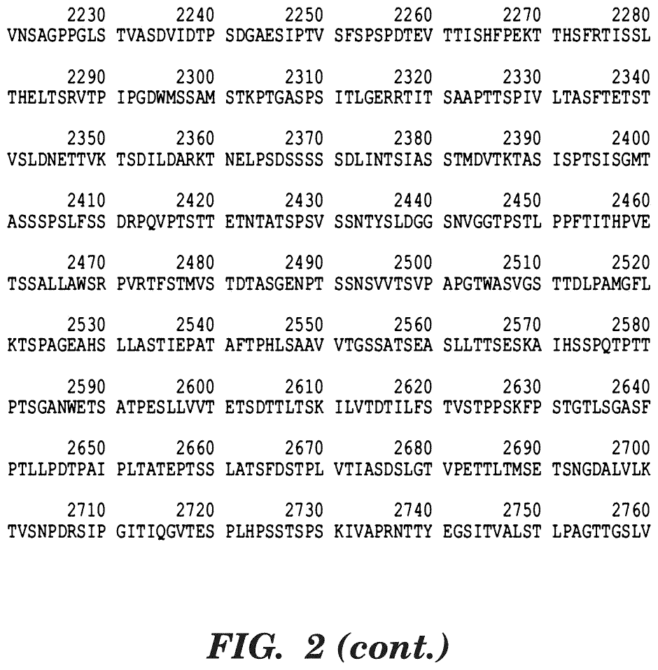

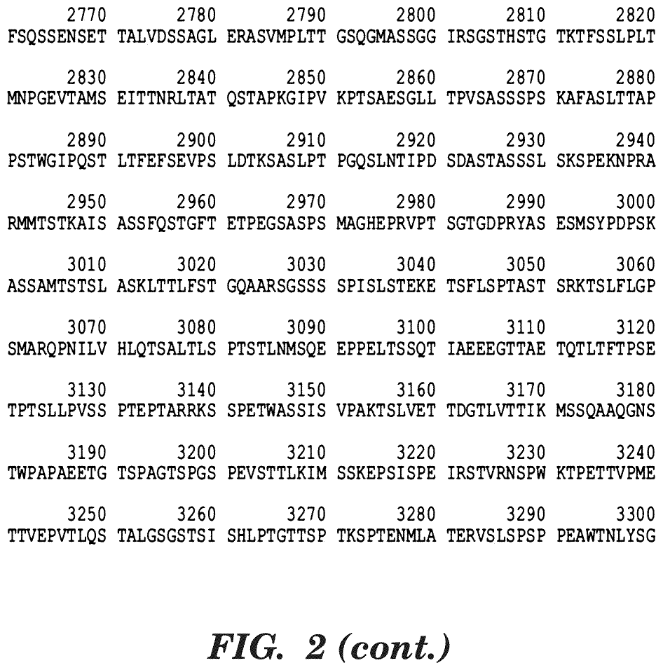

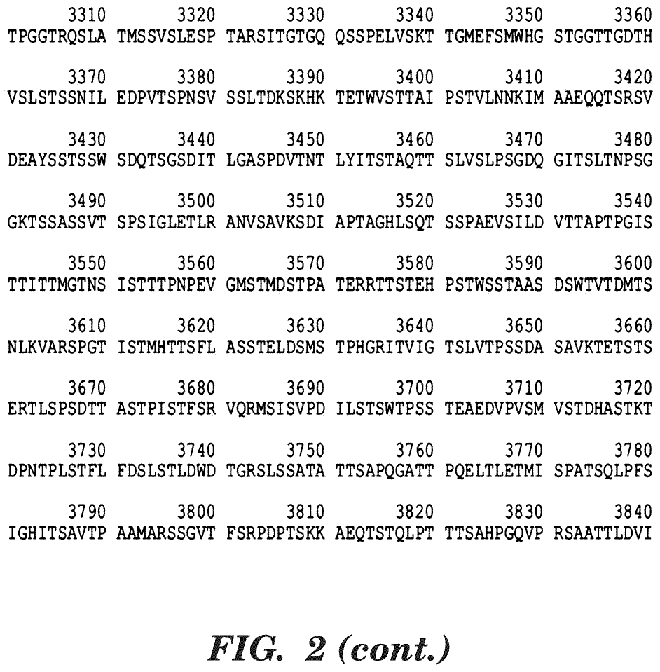

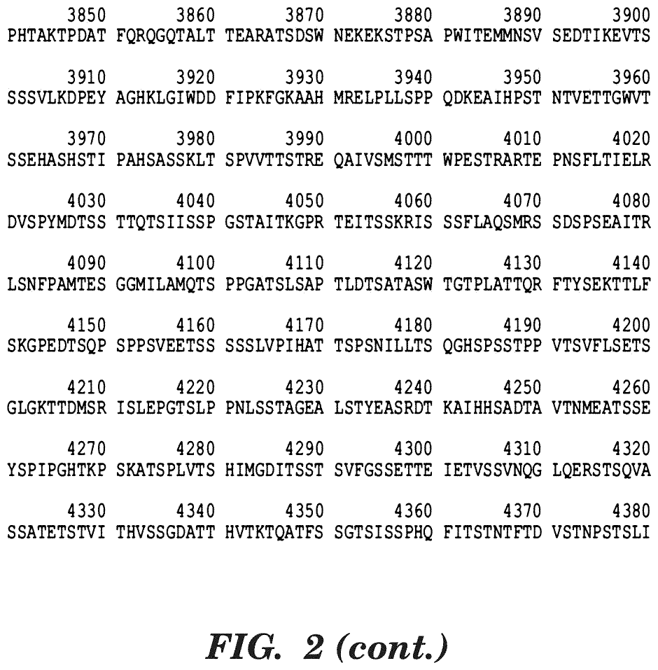

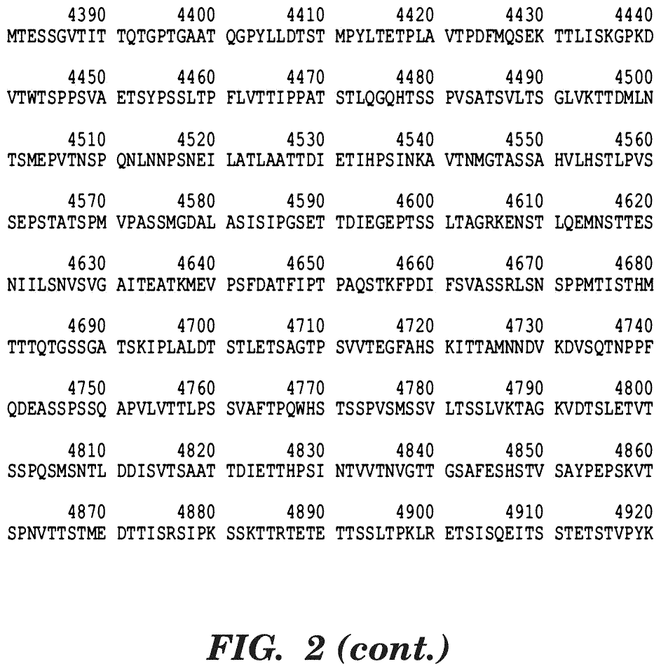

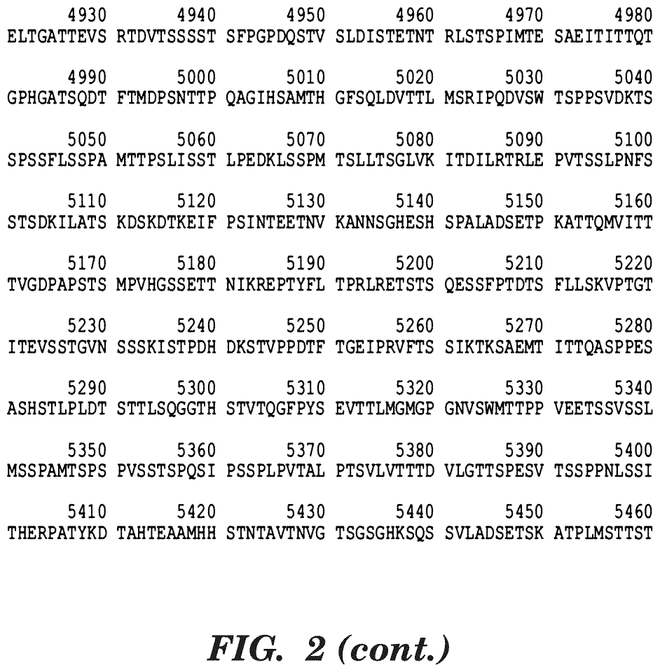

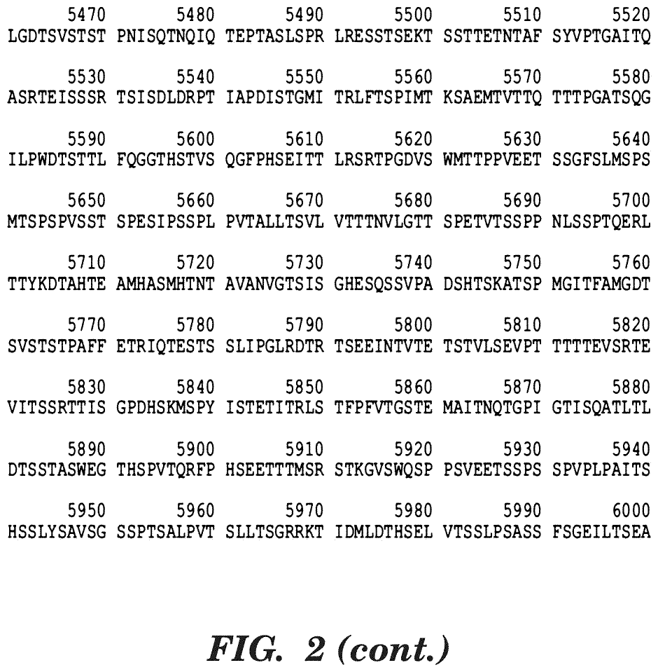

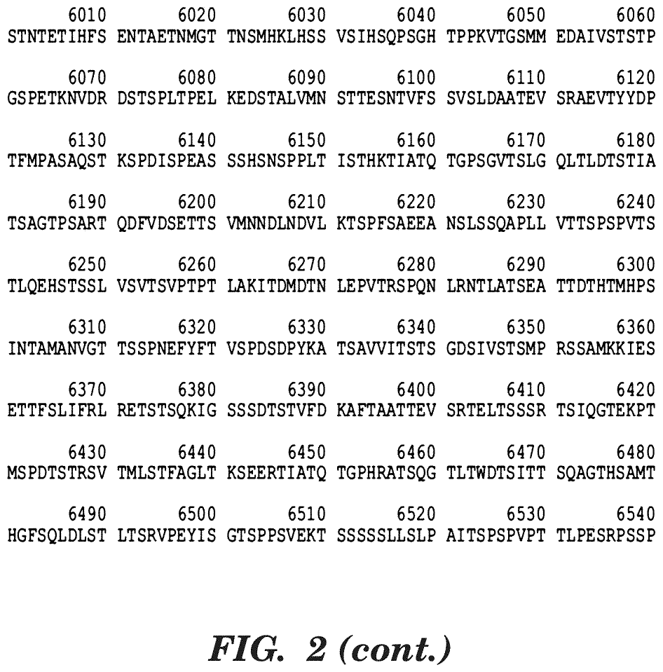

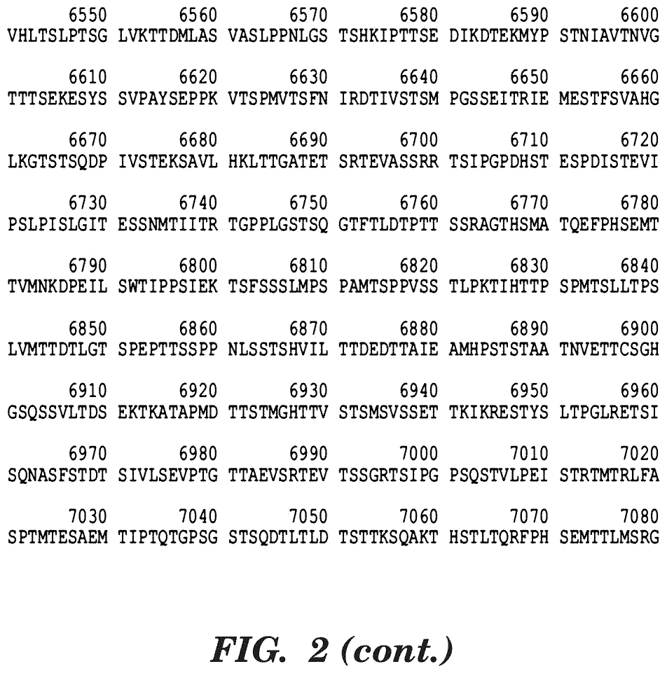

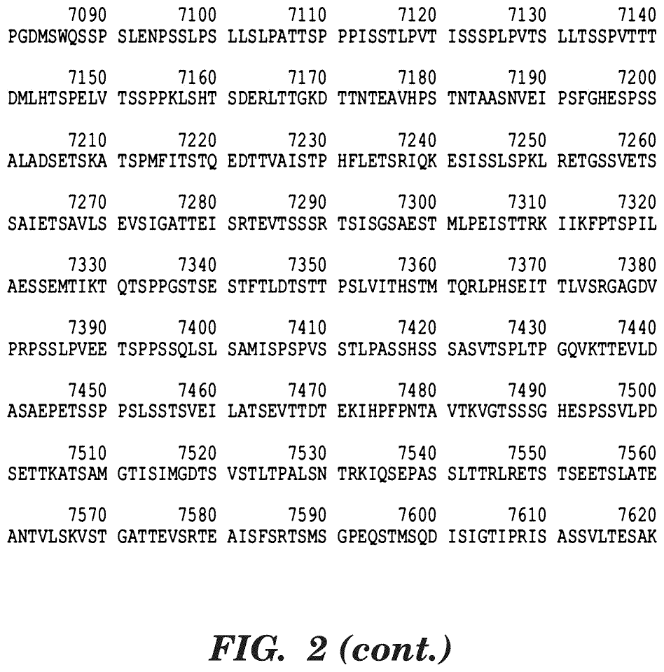

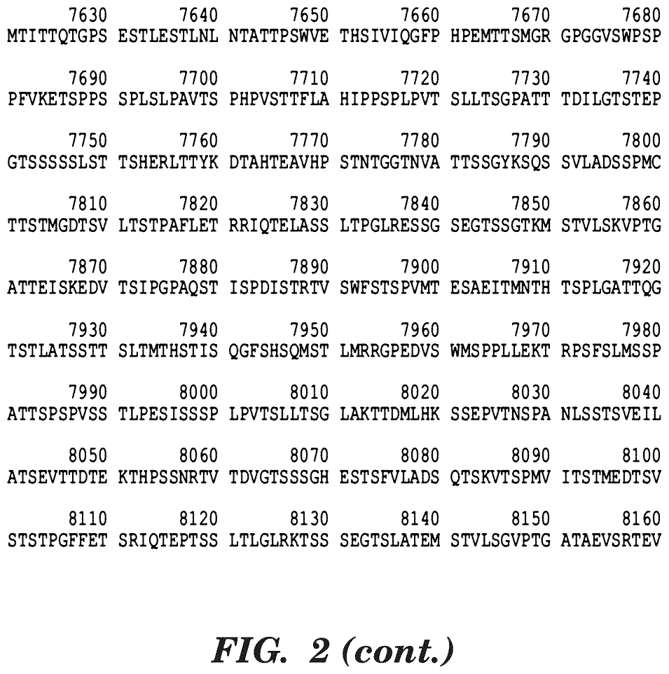

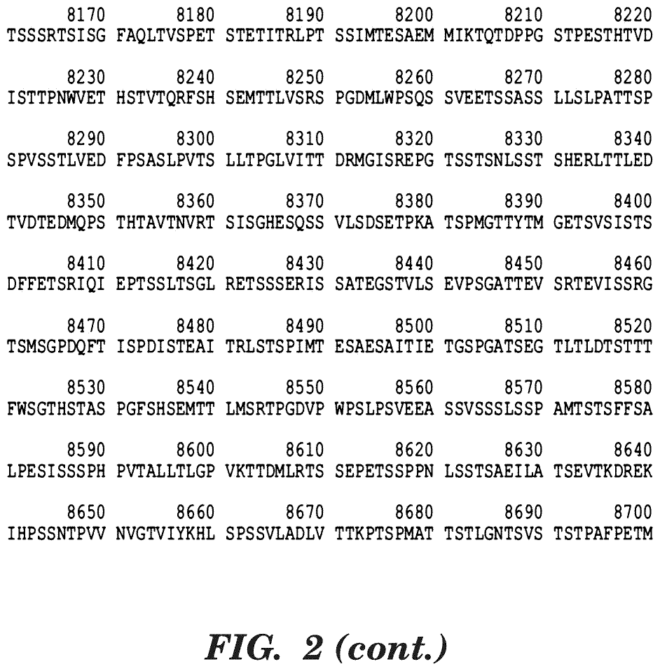

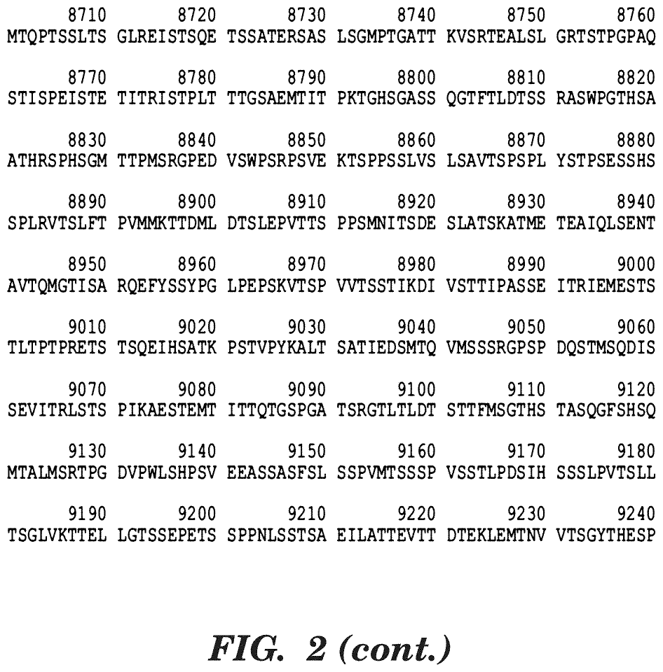

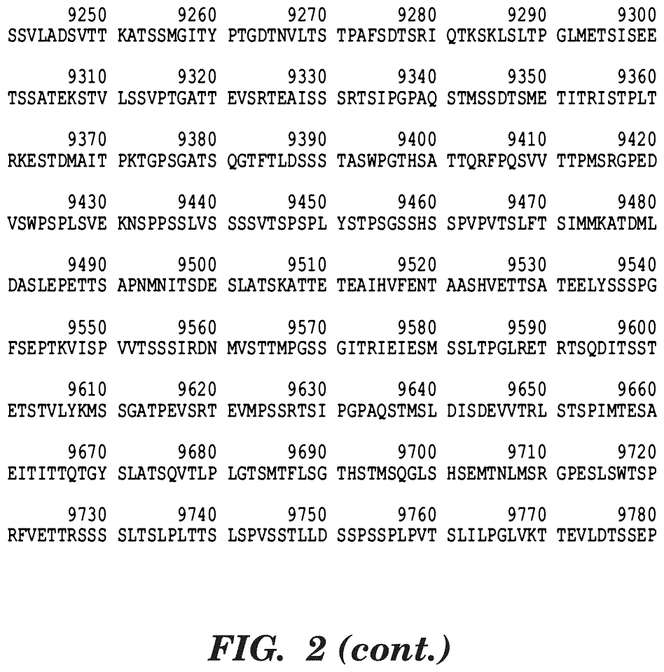

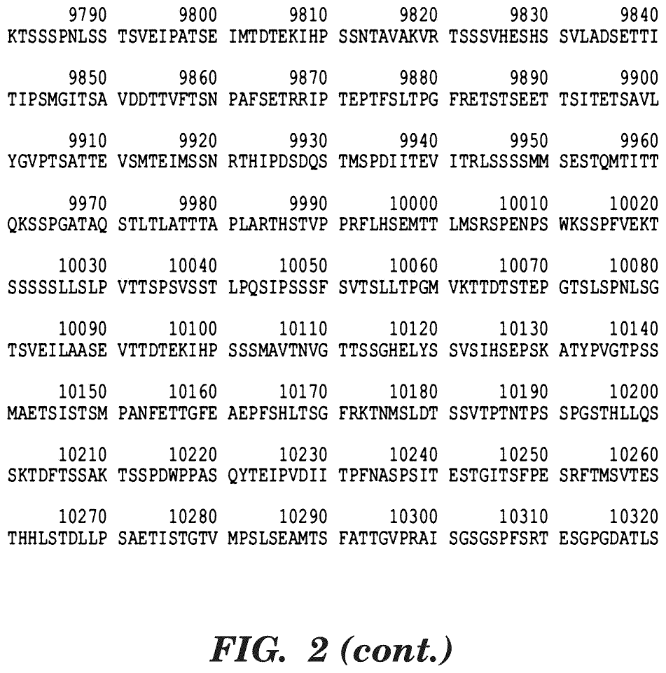

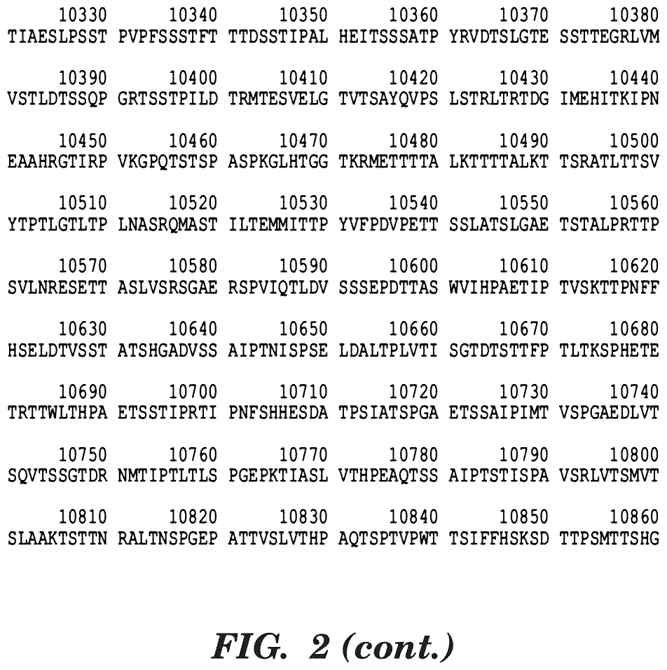

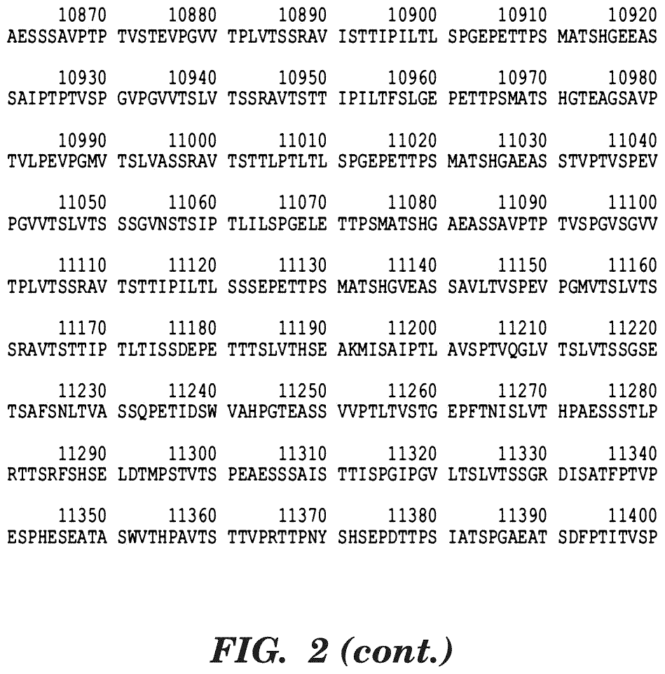

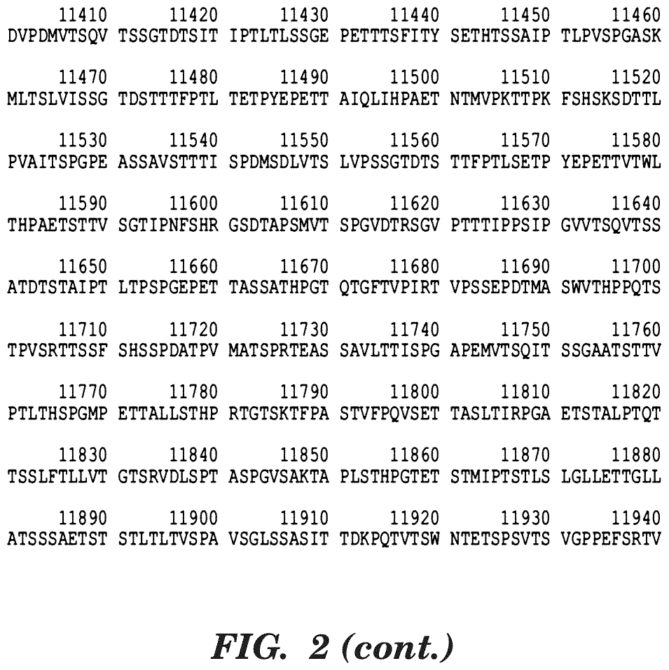

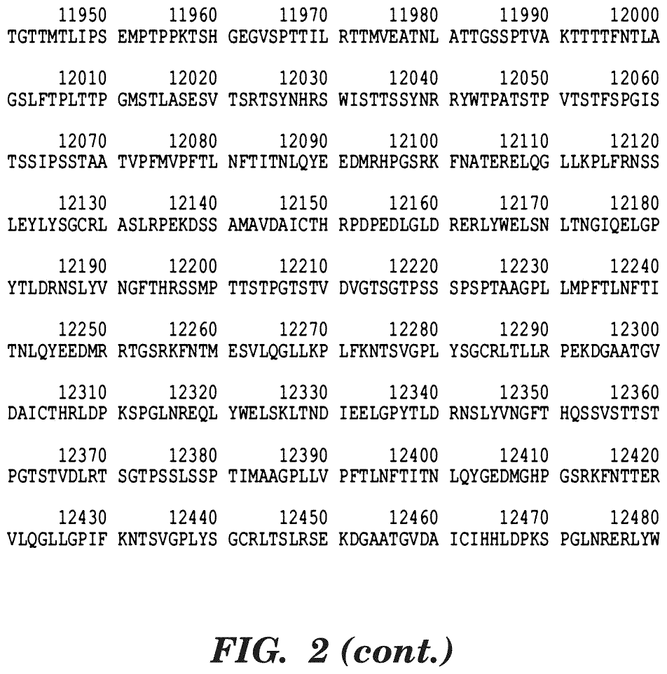

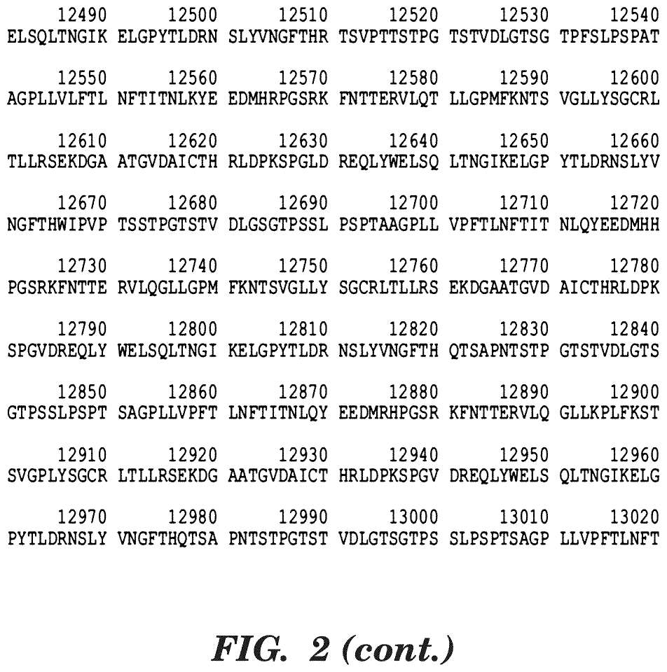

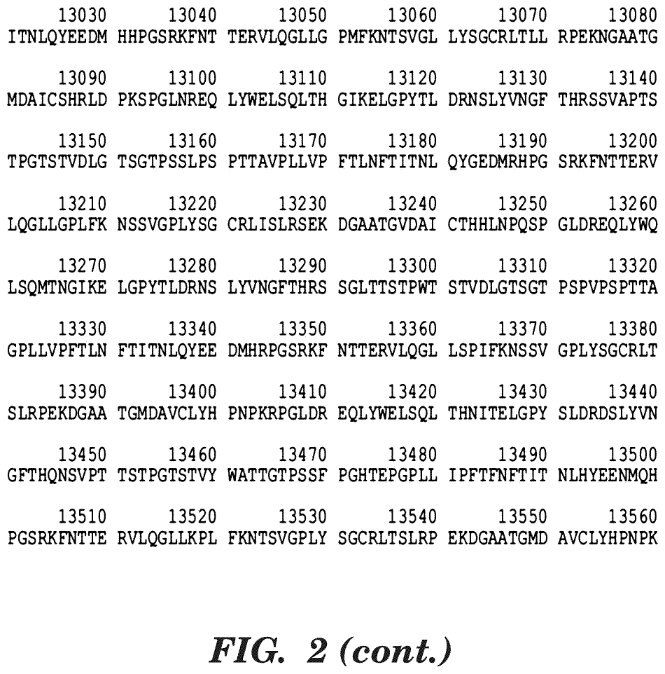

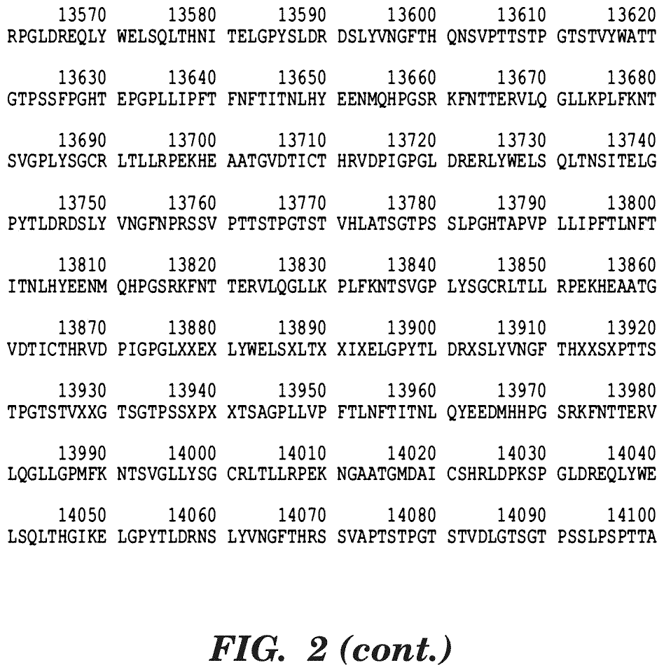

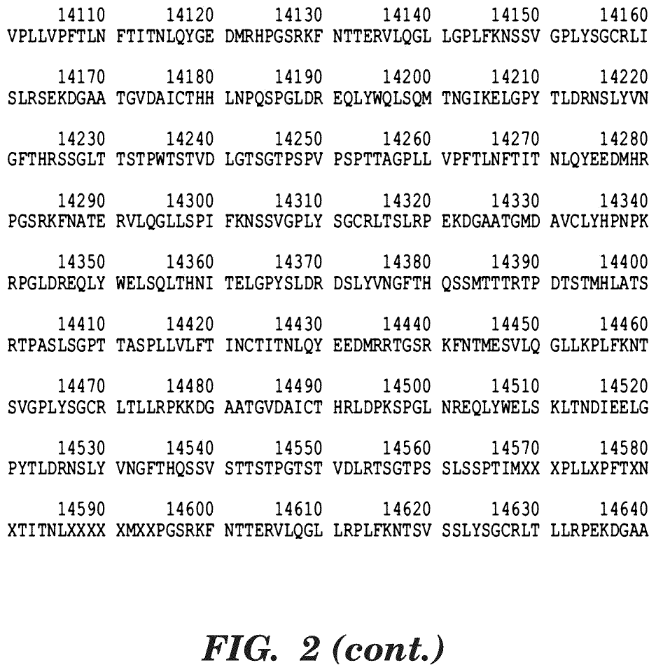

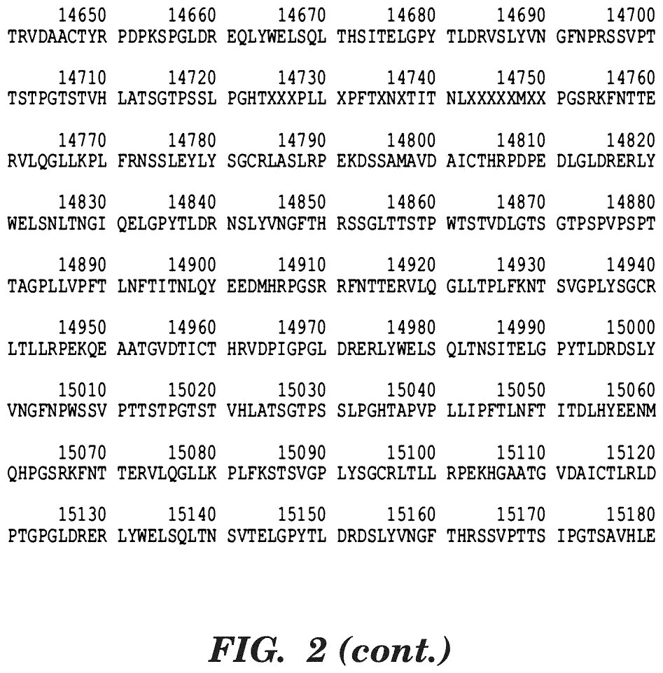

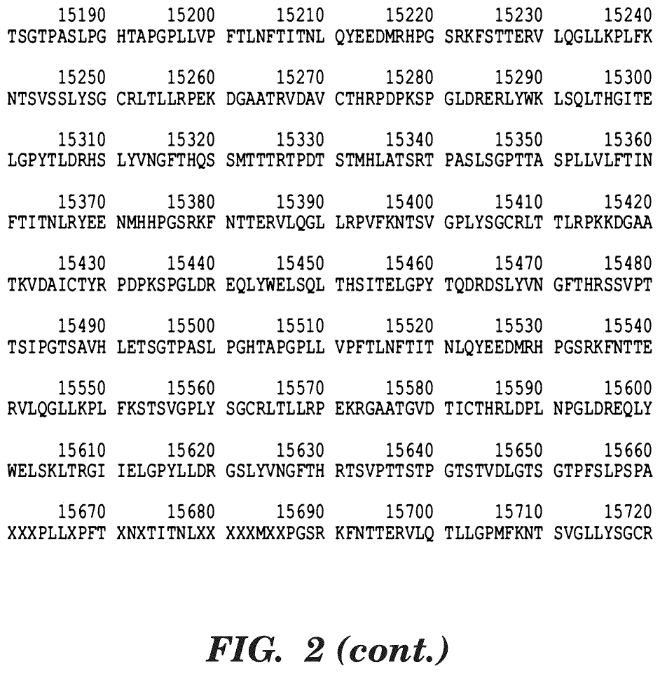

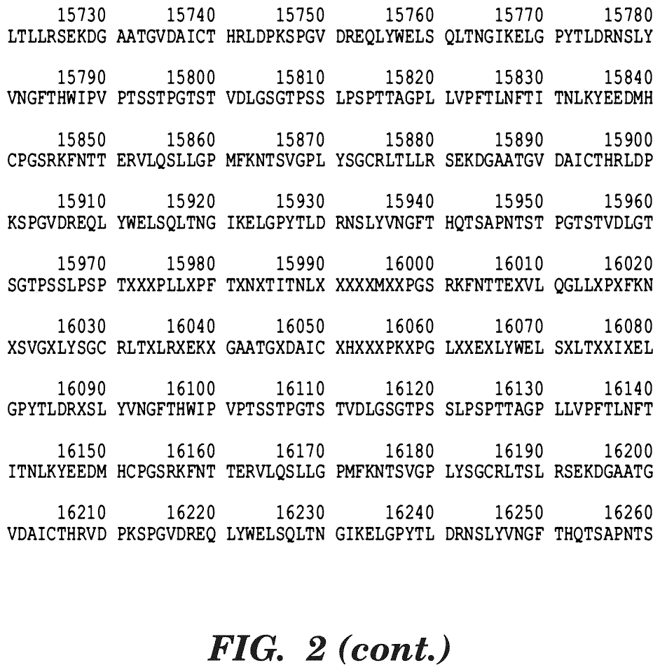

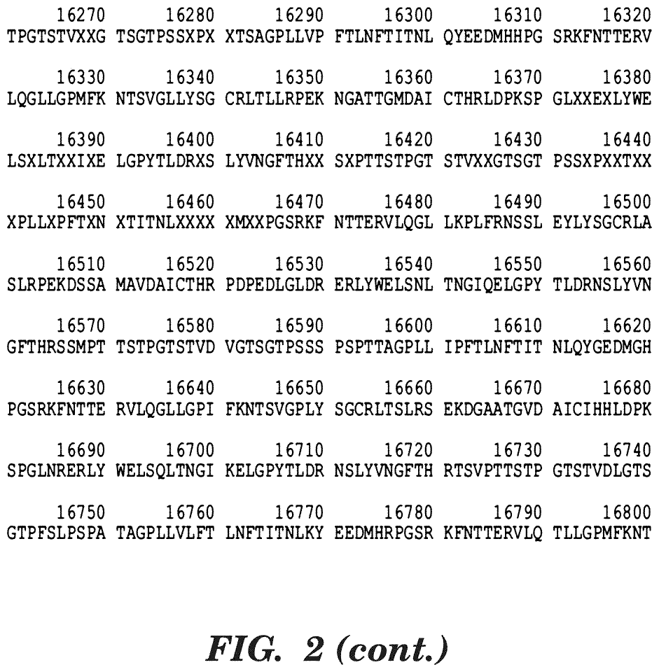

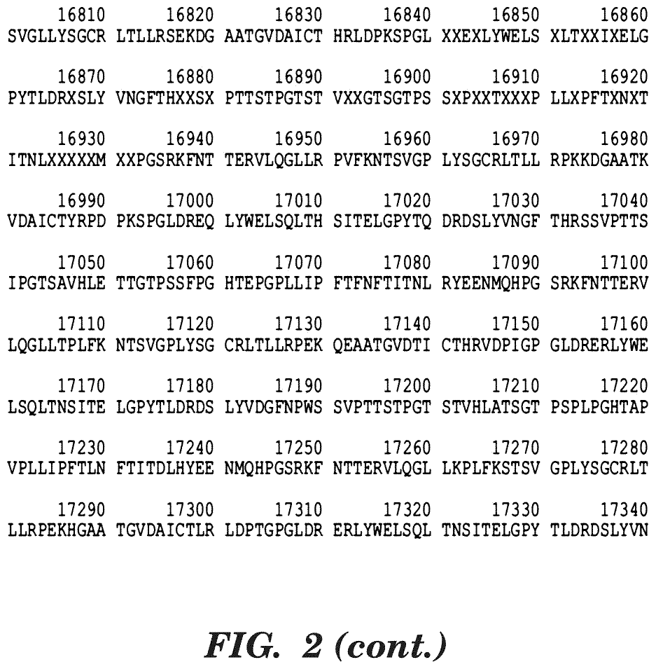

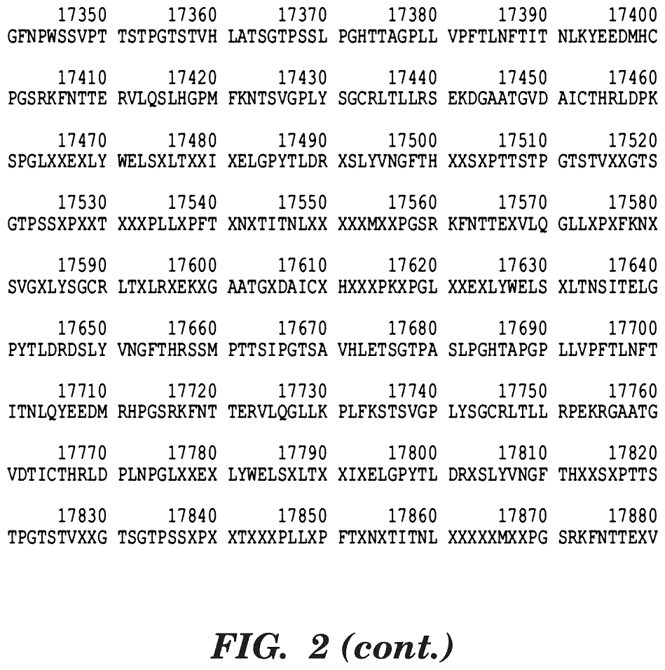

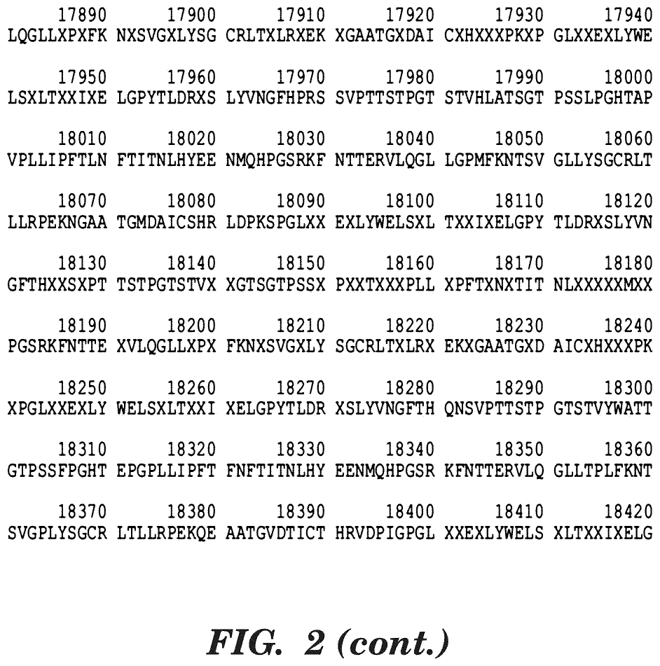

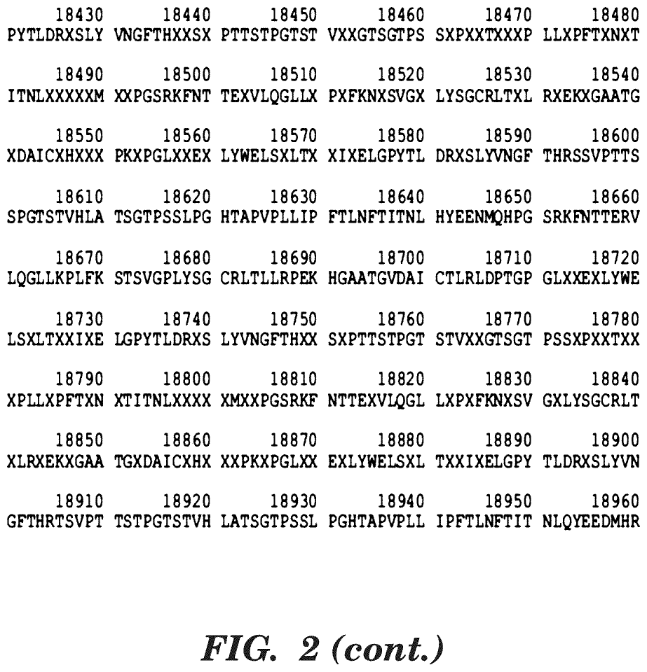

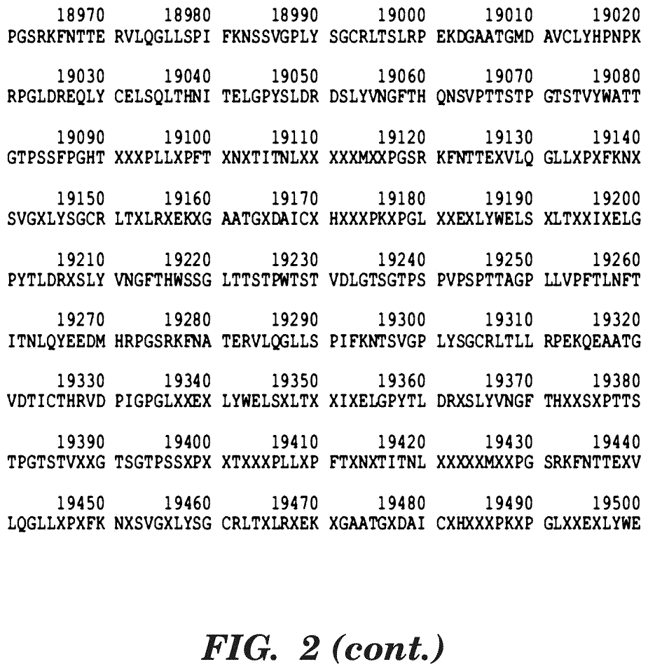

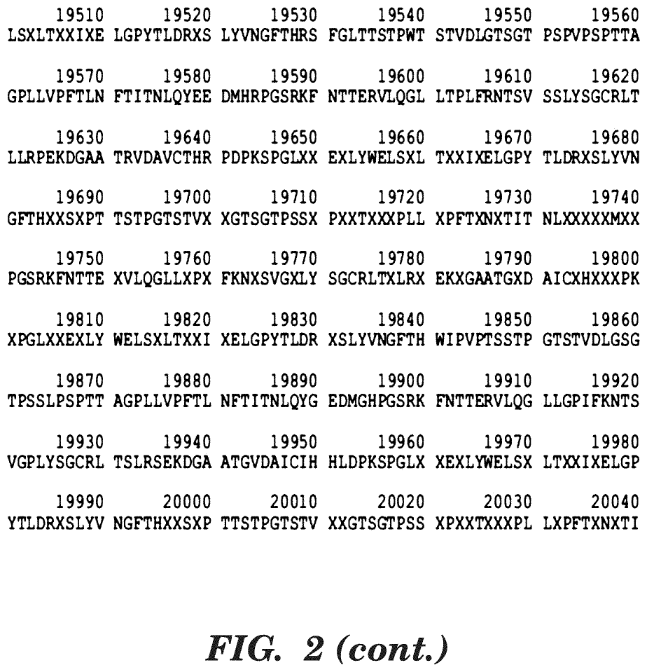

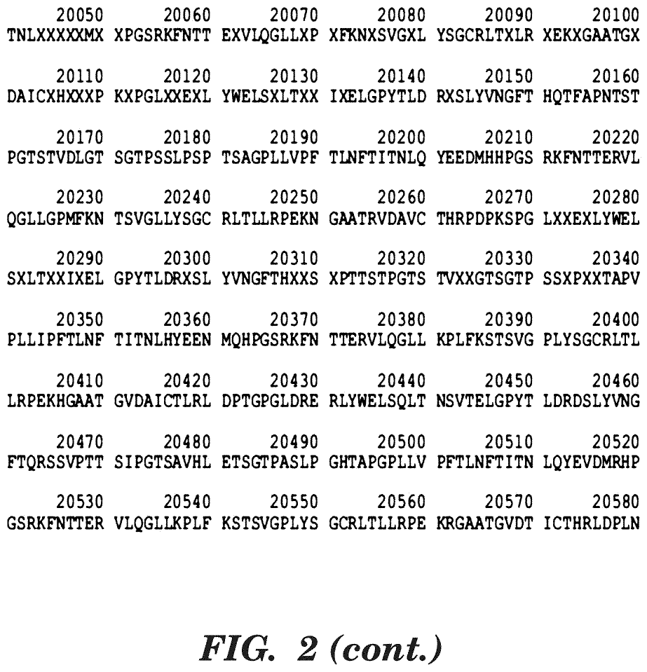

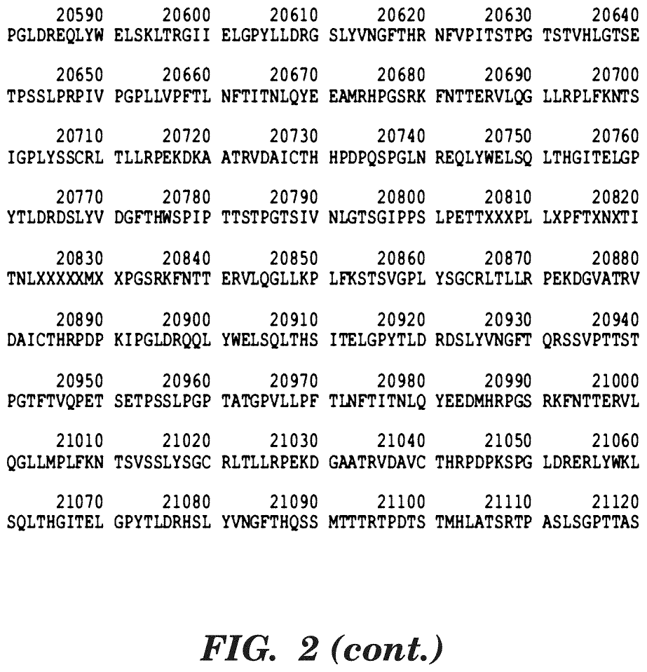

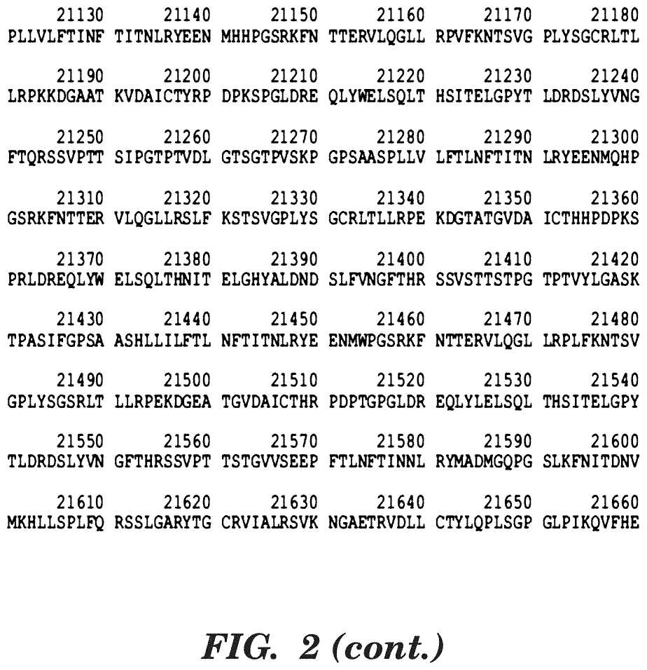

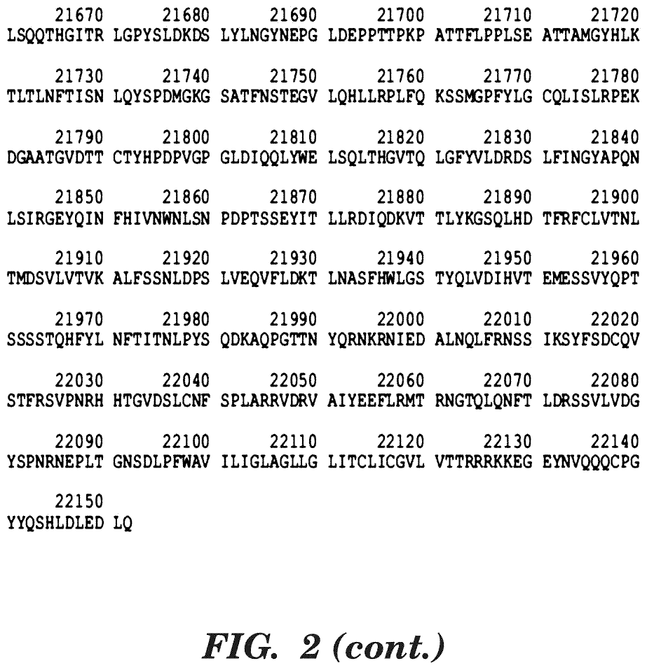

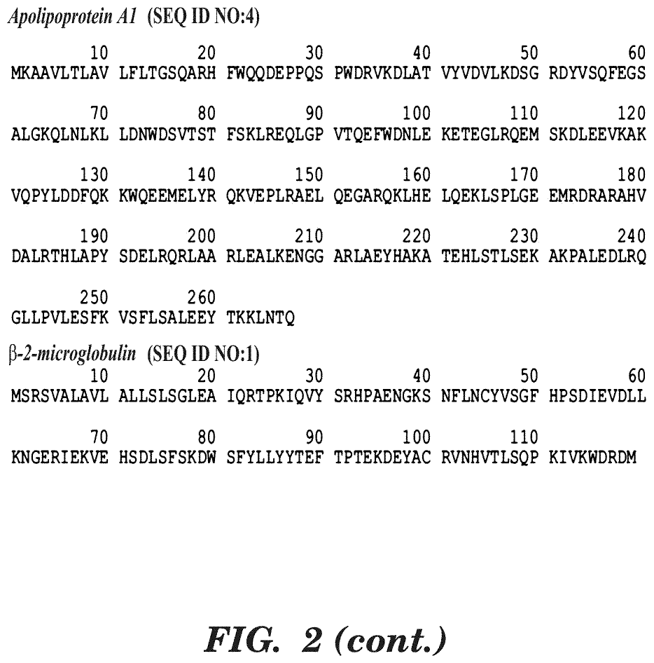

One exemplary biomarker present in the panel of the invention is FSH. FSH is a 128 amino acid protein (NCBI Accession number NP_000501). The amino acid sequence of an exemplary FSH polypeptide is set forth in FIG. 2. Antibodies to FSH can be made using any method well known in the art, or can be purchased from, for example, Santa Cruz Biotechnology, Inc. (e.g., Catalog Number sc-57149) (www.scbt.com, Santa Cruz, Calif.). In aspects of the invention, FSH is upregulated in subjects with ovarian cancer as compared to subjects that do not have ovarian cancer.

### Human Epididymis Protein 4 (HE4)

One exemplary biomarker present in the panel of the invention is HE4. HE4 is a 124 amino acid protein (NCBI Accession number NP_006094). The amino acid sequence of an exemplary HE4 polypeptide is set forth in FIG. 2. Antibodies to HE4 can be made using any method well known in the art, or can be purchased from, for example, Santa Cruz Biotechnology, Inc. (Catalog Number sc-27570) (www.scbt.com, Santa Cruz, Calif.). In aspects of the invention, HE4 is upregulated in subjects with ovarian cancer as compared to subjects that do not have ovarian cancer.

### Cancer Antigen 125 (CA 125)

One exemplary biomarker present in the panel of the invention is CA 125. CA 125 is a 22152 amino acid protein (Swiss-Prot Accession number Q8WXI7). The amino acid sequence of an exemplary CA 125 polypeptide is set forth in FIG. 2. Antibodies to CA 125 can be made using any method well known in the art, or can be purchased from, for example, Santa Cruz Biotechnology, Inc. (Catalog Number sc-52095) (www.scbt.com, Santa Cruz, Calif.). In aspects of the invention, CA 125 is upregulated in subjects with ovarian cancer as compared to subjects that do not have ovarian cancer.

### Transthyretin (Prealbumin)

Another exemplary biomarker present in the panel of the invention is a form of prealbumin, also referred to herein as transthyretin. Transthyretin is a 147 amino acid protein (Swiss Prot Accession number P02766). The amino acid sequence of an exemplary transthyretin polypeptide is set forth in FIG. 2. Antibodies to transthyretin can be made using any method well known in the art, or can be purchased from, for example, Santa Cruz Biotechnology, Inc. (Catalog Number sc-13098) (www.scbt.com, Santa Cruz, Calif.). In aspects of the invention, transthyretin is downregulated in subjects with ovarian cancer as compared to subjects that do not have ovarian cancer.

### Transferrin

Transferrin is another exemplary biomarker of the panel of biomarkers of the invention. Transferrin is a 698 amino acid protein (UniProtKB/TrEMBL Accession number Q06AH7). The amino acid sequence of an exemplary transferring polypeptide is set forth in FIG. 2. Antibodies to transferrin can be made using any method well known in the art, or can be purchased from, for example, Santa Cruz Biotechnology, Inc. (Catalog Number sc-52256) (www.scbt.com, Santa Cruz, Calif.). In aspects of the invention, transferrin is downregulated in subjects with ovarian cancer as compared to subjects that do not have ovarian cancer.

### Apolipoprotein A1

Apolipoprotein A1, also referred to herein as “Apo A1” is another exemplary biomarker in the panel of biomarkers of the invention. Apo A1 is a 267 amino acid protein (Swiss Prot Accession number P02647). The amino acid sequence of an exemplary Apo A1 is set forth in FIG. 2. Antibodies to Apolipoprotein A1 can be made using any method well known in the art, or can be purchased from, for example, Santa Cruz Biotechnology, Inc. (Catalog Number sc-130503) (www.scbt.com, Santa Cruz, Calif.). In aspects of the invention, Apo A1 is downregulated in subjects with ovarian cancer as compared to subjects that do not have ovarian cancer.

### β-2 Microglobulin

One exemplary biomarker that is useful in the methods of the present invention is β2-microglobulin. β2-microglobulin is described as a biomarker for ovarian cancer in US provisional patent publication 60/693,679, filed Jun. 24, 2005 (Fung et al.). The mature form of β2-microglobulin is a 99 amino acid protein derived from an 119 amino acid precursor (GI: 179318; SwissProt Accession No. P61769). The amino acid sequence of an exemplary β-2-microglobulin polypeptide is set forth in FIG. 2. The mature form of β-2-microglobulin consist of residues 21-119 of the β-2-microglobulin set forth in FIG. 2. β2-microglobulin is recognized by antibodies. Such antibodies can be made using any method well known in the art, and can also be commercially purchased from, e.g., Abcam (catalog AB759) (www.abcam.com, Cambridge, Mass.). In aspects of the invention, β2-microglobulin is upregulated in subjects with ovarian cancer as compared to subjects that do not have ovarian cancer.

### Biomarkers and Different Forms of a Protein

Proteins frequently exist in a sample in a plurality of different forms. These forms can result from pre- and/or post-translational modification. Pre-translational modified forms include allelic variants, splice variants and RNA editing forms. Post-translationally modified forms include forms resulting from proteolytic cleavage (e.g., cleavage of a signal sequence or fragments of a parent protein), glycosylation, phosphorylation, lipidation, oxidation, methylation, cysteinylation, sulphonation and acetylation. When detecting or measuring a protein in a sample, any or all of the forms may be measured to determine the level of biomarker or a form of interest is measured. The ability to differentiate between different forms of a protein depends upon the nature of the difference and the method used to detect or measure the protein. For example, an immunoassay using a monoclonal antibody will detect all forms of a protein containing the epitope and will not distinguish between them. However, a sandwich immunoassay that uses two antibodies directed against different epitopes on a protein will detect all forms of the protein that contain both epitopes and will not detect those forms that contain only one of the epitopes. Distinguishing different forms of an analyte or specifically detecting a particular form of an analyte is referred to as “resolving” the analyte.

Mass spectrometry is a particularly powerful methodology to resolve different forms of a protein because the different forms typically have different masses that can be resolved by mass spectrometry. Accordingly, if one form of a protein is a superior biomarker for a disease than another form of the biomarker, mass spectrometry may be able to specifically detect and measure the useful form where traditional immunoassay fails to distinguish the forms and fails to specifically detect to useful biomarker.

One useful methodology combines mass spectrometry with immunoassay. For example, a biospecific capture reagent (e.g., an antibody, aptamer, Affibody, and the like that recognizes the biomarker and other forms of it) is used to capture the biomarker of interest. In embodiments, the biospecific capture reagent is bound to a solid phase, such as a bead, a plate, a membrane or an array. After unbound materials are washed away, the captured analytes are detected and/or measured by mass spectrometry. This method will also result in the capture of protein interactors that are bound to the proteins or that are otherwise recognized by antibodies and that, themselves, can be biomarkers. Various forms of mass spectrometry are useful for detecting the protein forms, including laser desorption approaches, such as traditional MALDI or SELDI, electrospray ionization, and the like.

Thus, when reference is made herein to detecting a particular protein or to measuring the amount of a particular protein, it means detecting and measuring the protein with or without resolving various forms of protein. For example, the step of “detecting β-2 microglobulin” includes measuring β-2 microglobulin by means that do not differentiate between various forms of the protein (e.g., certain immunoassays) as well as by means that differentiate some forms from other forms or that measure a specific form of the protein.

### Detection of Biomarkers for Ovarian Cancer

The biomarkers of this invention can be detected by any suitable method. The methods described herein can be used individually or in combination for a more accurate detection of the biomarkers (e.g., biochip in combination with mass spectrometry, immunoassay in combination with mass spectrometry, and the like).

Detection paradigms that can be employed in the invention include, but are not limited to, optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are described infra.

### Detection by Immunoassay

In particular embodiments, the biomarkers of the invention are measured by immunoassay. Immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, Western blot, sandwich immunoassays including ELISA and other enzyme immunoassays, fluorescence-based immunoassays, chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In aspects of the invention, a sample is analyzed by means of a biochip (also known as a microarray). The polypeptides and nucleic acid molecules of the invention are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In aspects of the invention, a sample is analyzed by means of a protein biochip (also known as a protein microarray). Such biochips are useful in high-throughput low-cost screens to identify alterations in the expression or post-translation modification of a polypeptide of the invention, or a fragment thereof. In embodiments, a protein biochip of the invention binds a biomarker present in a subject sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, Calif.), Zyomyx (Hayward, Calif.), Packard BioScience Company (Meriden, Conn.), Phylos (Lexington, Mass.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In other embodiments, wash steps will occur at 68 C in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In aspects of the invention, the biomarkers of this invention are detected by mass spectrometry (MS). Mass spectrometry is a well known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-assisted Laser Desorption/Ionization (MALDI) and Electrospray Ionization (ESI)

In embodiments, the mass spectrometric technique for use in the invention is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on a collecting membrane, and the array may be analyzed. Subsequently, automated output of the results is provided by using an server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MS' analysis to be performed. Tandem mass spectrometry allows multiple reactions to be monitored at the same time.

Capillary infusion may be employed to introduce the marker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high performance liquid chromatography (HPLC) to a mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the invention is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's ProteinChip® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

### METHODS OF THE INVENTION

Panels comprising biomarkers of the invention are used to characterize a pelvic mass in a subject to determine whether the subject should be seen by a general surgeon or should be evaluated and/or treated by a gynecologic oncologist. In other embodiments, a panel of the invention is used to diagnose or stage an ovarian cancer by determining the molecular profile of the cancer. In certain embodiments, panels of the invention are used to select a course of treatment for a subject. The phrase “ovarian cancer status” includes any distinguishable manifestation of the disease, including non-disease. For example, ovarian cancer status includes, without limitation, the presence or absence of disease (e.g., ovarian cancer v. non-ovarian cancer), the risk of developing disease, the stage of the disease, the progression of disease (e.g., progress of disease or remission of disease over time), prognosis, the effectiveness or response to treatment of disease, and the determination of whether a pelvic mass is malignant of benign. Based on this status, further procedures may be indicated, including additional diagnostic tests or therapeutic procedures or regimens.

In aspects of the invention, the biomarkers of the invention can be used in diagnostic tests to identify early stage ovarian cancer in a subject.

The correlation of test results with ovarian cancer involves applying a classification algorithm of some kind to the results to generate the status. The classification algorithm may be as simple as determining whether or not the amounts of the markers listed in Table 1 are above or below a particular cut-off number. When multiple biomarkers are used, the classification algorithm may be a linear regression formula. Alternatively, the classification algorithm may be the product of any of a number of learning algorithms described herein.

In the case of complex classification algorithms, it may be necessary to perform the algorithm on the data, thereby determining the classification, using a computer, e.g., a programmable digital computer. In either case, one can then record the status on tangible medium, for example, in computer-readable format such as a memory drive or disk or simply printed on paper. The result also could be reported on a computer screen.

### Biomarkers of the Invention

Individual biomarkers are useful diagnostic biomarkers. In addition, as described in the examples, it has been found that a specific combination of biomarkers provides greater predictive value of a particular status than any single biomarker alone, or any other combination of previously identified biomarkers. Specifically, the detection of a plurality of biomarkers in a sample can increase the sensitivity, accuracy and specificity of the test.

Each biomarkers described herein can be differentially present in ovarian cancer, and, therefore, each is individually useful in aiding in the determination of ovarian cancer status. The method involves, first, measuring the selected biomarker in a subject, sample using any method well known in the art, including but not limited to the methods described herein, e.g. capture on a SELDI biochip followed by detection by mass spectrometry and, second, comparing the measurement with a diagnostic amount or cut-off that distinguishes a positive ovarian cancer status from a negative ovarian cancer status. The diagnostic amount represents a measured amount of a biomarker above which or below which a subject is classified as having a particular ovarian cancer status. For example, if the biomarker is up-regulated compared to normal during ovarian cancer, then a measured amount above the diagnostic cutoff provides a diagnosis of ovarian cancer. Alternatively, if the biomarker is down-regulated during ovarian cancer, then a measured amount below the diagnostic cutoff provides a diagnosis of ovarian cancer. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different ovarian cancer statuses, as was done here, and drawing the cut-off to suit the diagnostician's desired levels of specificity and sensitivity.

The biomarkers of this invention (used alone or in combination) show a statistical difference in different ovarian cancer statuses of at least p≤0.05, p≤10⁻², p≤10⁻³, p≤10⁻⁴, or p≤10′. Diagnostic tests that use these biomarkers alone or in combination show a sensitivity and specificity of at least 75%, at least 80%, at least 85%, at least 90%, at least 95%, at least 98%, or about 100%.

### Determining Course (Progression/Remission) of Disease

In one embodiment, this invention provides methods for determining the course of disease in a subject. Disease course refers to changes in disease status over time, including disease progression (worsening) and disease regression (improvement). Over time, the amounts or relative amounts (e.g., the pattern) of the biomarkers change. Accordingly, this method involves measuring the panel of biomarkers in a subject at least two different time points, e.g., a first time and a second time, and comparing the change in amounts, if any. The course of disease (e.g., during treatment) is determined based on these comparisons.

### Reporting the Status

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the differential presence or absence in a test subject of the biomarkers of Table 1 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### Subject Management

In certain embodiments, the methods of the invention involve managing subject treatment based on the status. Such management includes referral, for example, to a gynecologic oncologist, or other actions of the physician or clinician subsequent to determining ovarian cancer status. For example, if a physician makes a diagnosis of ovarian cancer, then a certain regime of treatment, such as prescription or administration of therapeutic agent might follow. Alternatively, a diagnosis of non-ovarian cancer or non-ovarian cancer might be followed with further testing to determine a specific disease that might the patient might be suffering from. Also, if the diagnostic test gives an inconclusive result on ovarian cancer status, further tests may be called for.

In one embodiment, the diagnosis may be determining if a pelvic mass is benign or malignant. If the diagnosis is malignant, a gynecologic oncologist may be chosen to perform the surgery. In contrast, if the diagnosis is benign, a general surgeon or a gynecologist may be chosen to perform the surgery.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

### Hardware and Software

The any of the methods described herein, the step of correlating the measurement of the biomarker(s) with ovarian cancer can be performed on general-purpose or specially-programmed hardware or software.

In aspects, the analysis is performed by a software classification algorithm. The analysis of analytes by any detection method well known in the art, including, but not limited to the methods described herein, will generate results that are subject to data processing. Data processing can be performed by the software classification algorithm. Such software classification algorithms are well known in the art and one of ordinary skill can readily select and use the appropriate software to analyze the results obtained from a specific detection method.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

For example, analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. Exemplary software includes, but is not limited to, Ciphergen's ProteinChip® software, in which data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, for example, as part of Ciphergen's ProteinChip® software package, that can automate the detection of peaks. This software functions by identifying signals having a signal-to-noise ratio above a selected threshold and labeling the mass of the peak at the centroid of the peak signal. In embodiments, many spectra are compared to identify identical peaks present in some selected percentage of the mass spectra. One version of this software clusters all peaks appearing in the various spectra within a defined mass range, and assigns a mass (N/Z) to all the peaks that are near the mid-point of the mass (M/Z) cluster.

In aspects, software used to analyze the data can include code that applies an algorithm to the analysis of the results (e.g., signal to determine whether the signal represents a peak in a signal that corresponds to a biomarker according to the present invention). The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

### Classification Algorithms for Qualifying Ovarian Cancer Status

In some embodiments, data derived from the assays (e.g., ELISA assays) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In embodiments, a supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for ovarian cancer. The classification algorithms, in turn, form the base for diagnostic tests by providing diagnostic values (e.g., cut-off points) for biomarkers used singly or in combination.

### Kits for Detection of Biomarkers for Ovarian Cancer

In another aspect, the invention provides kits for aiding in the diagnosis of ovarian cancer (e.g., identifying ovarian cancer status, detecting ovarian cancer, identifying early stage ovarian cancer, selecting a treatment method for a subject at risk of having ovarian cancer, and the like), which kits are used to detect biomarkers according to the invention. In one embodiment, the kit comprises agents that specifically recognize the biomarkers identified in Table 1. In related embodiments, the agents are antibodies. The kit may contain 1, 2, 3, 4, 5, or more different antibodies that each specifically recognize one of the biomarkers set forth in Table 1.

In another embodiment, the kit comprises a solid support, such as a chip, a microtiter plate or a bead or resin having capture reagents attached thereon, wherein the capture reagents bind the biomarkers of the invention. Thus, for example, the kits of the present invention can comprise mass spectrometry probes for SELDI, such as ProteinChip® arrays. In the case of biospecific capture reagents, the kit can comprise a solid support with a reactive surface, and a container comprising the biospecific capture reagents.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for use in any of the methods described herein. In embodiments, the instructions provide suitable operational parameters in the form of a label or separate insert. For example, the instructions may inform a consumer about how to collect the sample, how to wash the probe or the particular biomarkers to be detected.

In yet another embodiment, the kit can comprise one or more containers with controls (e.g., biomarker samples) to be used as standard(s) for calibration.

### Example 1: Ovarian Cancer in African Americans

No data exists on sensitivity of CA125 to detect ovarian malignancy based on ethnicity. We compared CA125 to Multivariate Index Assay (MIA) (OVA1®) in evaluation of different populations with adnexal masses. Serum values of CA125 and the Multivariate Index Assay (MIA) (OVA1®), which measures Transthyretin (TT or prealbumin), Apolipoprotein A-1 (Apo A-1), beta 2-Microglobulin (beta 2M), Transferrin (Tfr) and Cancer Antigen 125 (CA 125 II), were evaluated in subgroups of women who underwent surgery for adnexal masses. We determined sensitivity for detection of ovarian malignancy in these subgroups.

Samples were also reviewed from previous prospective studies validating OVA1®. Of these 250 women were Caucasian (C) and 24 African-American (AA). In premenopausal women positive OVA1® testing was above 5.0 and CA125 above 200. In postmenopausal women, positive OVA1® was above 4.4 and CA125 above 35. Sensitivity for preoperative test results were calculated using DTCompair package.

Primary ovarian malignancy was diagnosed in 179 cases (167 Caucasian (C)/12 African American (AA)), metastatic disease to the ovary in an additional 27 cases (22C/5AA). Borderline or low malignant potential tumors accounted for 52 patients (47C/5AA). Non-primary tumors with no ovarian involvement were found in 16 women (14C/2AA).

Sensitivity testing results are seen for each subgroup and for OVA1® levels and CA125 results:

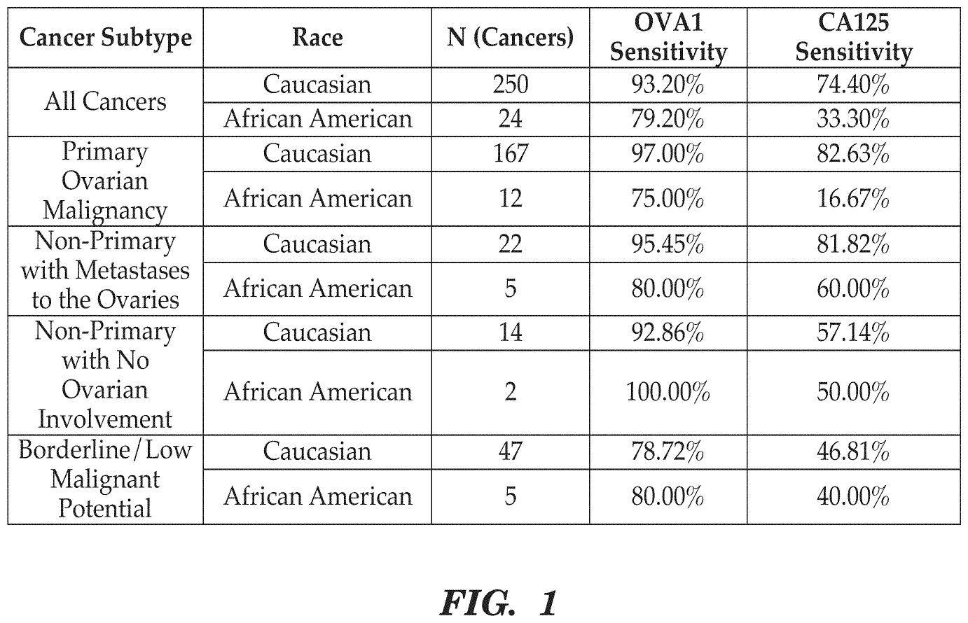

### All Cancers: Caucasian OVA1® 93.20%, CA125 74.4%, AA OVA1® 79.2% CA125 33.3%. Primary Ovarian Malignancy: Caucasian OVA1® 97.0%, CA125 82.63%, AA OVA1® 75.0% CA125 16.67%. See FIG. 1.

Additionally, in primary ovarian malignancy data based on pre- and post-menopausal status in the AA population reflect similar results. Sensitivity of OVA1® in premenopausal AA women was 77.8% and CA125 only 11.1%. In postmenopausal AA women sensitivity of OVA1® was 66.7% and CA125 33.3%.

Our results demonstrate that CA125 in AA women with adnexal masses have lower sensitivity for detection of malignancy than OVA1®. This is true across all cancers detected but is most pronounced pre-menopausal AA women. Implementation of OVA1® in evaluation of adnexal masses will increase sensitivity of detection of malignancy over CA125 testing with most marked results in AA women. In particular, these results indicate that the cut-off values indicating that an AA woman has ovarian cancer should be lowered to ensure that the test has an acceptable level of sensitivity.

Markers of ethnicity are known in the art and are described, for example, ancestry is estimated using ancestry informative markers (AIMs), which are a set of genetic variations for a particular DNA sequence that appear in different frequencies in populations from different regions of the world. The use of AIMs compares an individual's polymorphisms at these markers with previously analyzed genomic reference sets from people whose ancestral history is fairly well known. AIMs are used to estimate the geographical origins of an individual's ancestors, typically expressed as proportions of one's ancestry that comes from different continental regions.

Availability of genetic markers that are ancestry informative and newly developed statistical methods may overcome concerns regarding race/ethnicity categorization. There is evidence that measures of genetic ancestry can improve clinical care for people of mixed race. For example, physicians assessing ovarian cancer can make more accurate diagnoses when they use a reference standard from the patients' actual genetic ancestry than self-reported race or ethnicity. Genetic markers used to infer ancestry include autosomal SNPs, Y-SNPs, mitochondrial SNPs, and X-SNPs.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents, publications, and accession numbers mentioned in this specification are herein incorporated by reference to the same extent as if each independent patent, publication, and accession number was specifically and individually indicated to be incorporated by reference.

1. A panel for pre-operatively assessing an African American subject's risk of having ovarian cancer, the panel comprising markers:
(a) Transthyretin (TT or prealbumin), Apolipoprotein A-1 (Apo A-1), beta 2-Microglobulin (beta 2M), Transferrin (Tfr), Cancer Antigen 125 (CA 125), and a marker of ethnicity;
(b) apolipoprotein A-1, CA 125, follicle stimulating hormone, human epididymis protein 4, transferrin, and a marker of ethnicity; or
(c) CA 125 and a marker of ethnicity; or a set of capture reagents each of which specifically binds a marker from among panels a-c.

2. The panel of claim 1, wherein the capture reagents are antibodies that selectively bind the markers.

3. A method for pre-operatively assessing an African American subject's risk of having ovarian cancer, the method comprising detecting binding of a capture reagent to a marker selected from a panel comprising markers:
(a) Transthyretin (TT or prealbumin), Apolipoprotein A-1 (Apo A-1), beta 2-Microglobulin (beta 2M), Transferrin (Tfr), Cancer Antigen 125 (CA 125), and a marker of ethnicity;
(b) apolipoprotein A-1, CA 125, follicle stimulating hormone, human epididymis protein 4, transferrin, and a marker of ethnicity; or
(c) CA 125 and a marker of ethnicity.

4. The method of claim 3, wherein the detecting is by immunoassay or affinity capture.

5. The method of claim 4, wherein the immunoassay comprises affinity capture assay, immunometric assay, heterogeneous chemiluminscence immunometric assay, homogeneous chemiluminscence immunometric assay, ELISA, western blotting, radioimmunoassay, magnetic immunoassay, real-time immunoquantitative PCR (iqPCR) and SERS label free assay.

6. The method of claim 3, wherein the method is carried out in a plate, chip, beads, microfluidic platform, membrane, planar microarray, or suspension array.

7. The panel of claim 1, wherein the panel is fixed to a substrate.

8. The panel of claim 7, wherein the substrate is a plate, chip, beads, microfluidic platform, membrane, planar microarray, or suspension array.

9. The panel of claim 1, wherein the marker of ethnicity identifies the subject as an African American.

10. The panel of claim 1, wherein the marker of ethnicity is a single nucleotide polymorphism.

11. The method of claim 3, wherein the marker of ethnicity identifies the subject as an African American.

12. The method of claim 3, wherein the marker of ethnicity is a single nucleotide polymorphism.
